(19) United States
(12) Patent Application Publication
(10) Pub. No.: US 2007/0233108 A1
(43) Pub. Date: Oct. 4, 2007

(54) Spine fixation device

(76) Inventors: Gregory C. Stalcup, Columbia City, IN (US); Jeff D. Gordon, Seattle, WA (US)

Correspondence Address: WOOD, HERRON & EVANS (ZIMMER SPINE), 2700 CAREW TOWER, 441 VINE STREET, CINCINNATI, OH, 45202, US

(21) Appl. No.: 11/375,993

(22) Filed: Mar. 15, 2006

(51) Int. Cl. A61F 2/30

(52) U.S. Cl. 606/69

(57) ABSTRACT

An orthopedic fixation device comprises a plate defining a longitudinal axis, the plate being configured to be affixed to the spine with the longitudinal axis generally aligned with the spinal column and comprising an upper edge and a lower edge positioned apart along the longitudinal axis, and a top surface and a bottom surface separated by the upper and lower edges, at least one of the edges being generally oblique to the longitudinal axis.

### TECHNICAL FIELD

The invention relates to spinal fixation systems. More particularly, the invention relates to an improved modular fixation plate system.

### BACKGROUND

Spinal fixation devices and techniques by which adjacent vertebrae are fused together and/or linked by rigid plates or rods are known. For example, in a traditional multi-level fixation technique, a single plate extending across two or more levels, with bone screw holes positioned at each vertebra involved, can be used. Alternatively, multiple single-level plates can be affixed to the vertebrae in an end-to-end fashion.

While the traditional spinal plates can be effective in many situations, there is still a need for additional types of fixation devices that offer additional spinal treatment capabilities. For example, unitary multi-level plates may not be appropriate in some cases because certain types of vertebral malformations may render it undesirable to affix the plate at certain places. Similarly, in spinal fixation by multiple single-level plates, it may become undesirable to place certain plates at certain positions along the longitudinal directions of the spinal column. Traditional fixation plate configurations provide only limited positional adjustability, which can result in sub-optimal placement of the plates. Additionally, traditional fixation plates do not readily accommodate atypical vertebral shapes, such as those encountered in patients whom have had portions of one or more vertebra surgically removed. Thus, there is a need in the art for a spinal fixation plate that provides optimal positional adjustability and that can readily accommodate and conform to atypical vertebral shapes and configurations.

### SUMMARY

The invention disclosed herein is aimed at providing an improved method and apparatus of spinal fixation by spinal plates. In one embodiment of the invention, an orthopedic fixation device comprises a plate defining a longitudinal axis that is configured to be affixed to at least two separate vertebrae with the longitudinal axis generally aligned with the spinal column. The plate includes an upper edge and a lower edge positioned apart along the longitudinal axis, and a top surface and a bottom surface separated by the upper and lower edges. At least one of the edges is generally oblique to the longitudinal axis. In another embodiment, the plate additionally has holes, at least two of which are near the upper edge and at least two others are near the lower edge. The holes near the edge are generally oblique to the longitudinal axis and are positioned along a line that is also generally oblique to the longitudinal axis.

In a further embodiment, a method of spinal fixation comprises affixing two or more plates described above to three or more consecutive vertebrae with the longitudinal axes of the plates generally parallel to the spinal column but offset from each other. The plates may also be positioned such that the total minimum height of the plates along the spinal column is smaller than the minimum height attainable when the longitudinal axes of the plates are aligned with no offset.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side elevation view of a human spinal column;

FIGS. 2 and 3 show anterior-posterior and lateral schematic views, respectively, of a portion of the spinal column to which a plurality of fixation plates are affixed according to one embodiment of the present invention;

FIG. 4 is a rear elevational view of a fixation plate according to one embodiment of the present invention;

FIG. 5 is a side view of the fixation plate of FIG. 4;

FIGS. 6(a) and 6(b) are rear elevational views of the fixation plates and spine shown in FIG. 2;

FIG. 7 is top view of a fixation plate according to another embodiment of the present invention;

FIG. 8 is a perspective view of the fixation plate shown in FIG. 7;

FIG. 9 is an end view of the fixation plate shown in FIG. 7;

FIG. 10(a) is a side view of the fixation plate shown in FIG. 7;

FIG. 10(b) is an enlarged view of Detail A of the screw locking member of the fixation plate shown in FIG. 7;

FIGS. 11 and 12 are side views of additional embodiments of the present invention.

### DETAILED DESCRIPTION OF SPECIFIC EMBODIMENTS

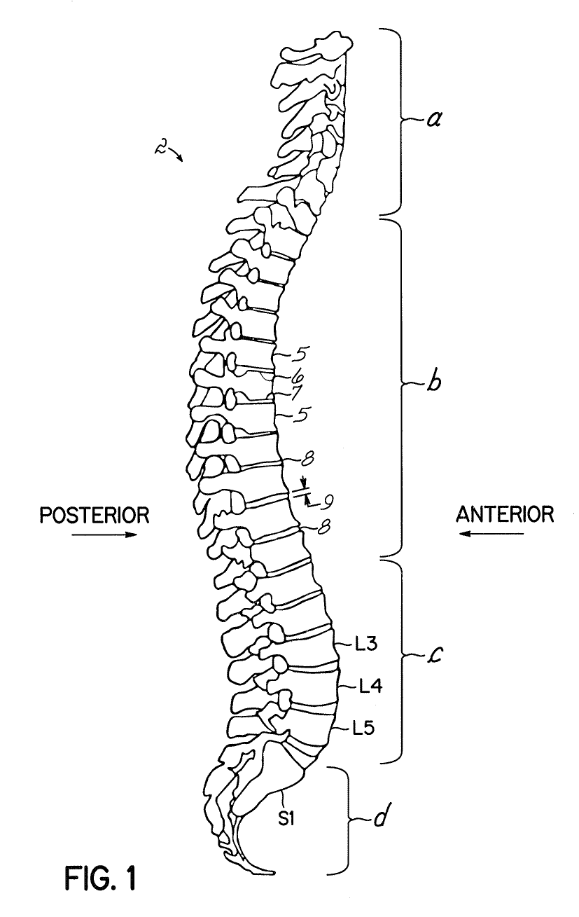

FIG. 1 illustrates a human spinal column 2 including vertebrae 5 belonging to one of a cervical region a, a thoracic region b, a lumbar region c and a sacral region d of the spinal column 2. Each vertebra 5 includes a superior end plate 6 and an inferior end plate 7. Intervertebral discs 8 are positioned in intervertebral spaces 9 between adjacent vertebrae 5. The individual vertebra in each of the regions a, b and c may be uniquely identified such as by references L3, L4 and L5 for vertebrae in the lumbar region c, and S1 for the vertebra in the sacral region d. The term “anterior spine” refers to the portion of the spinal column 2 nearer the front of the body, as distinguished from the posterior spine, which is the portion of the spinal column 2 nearer the back of the body.

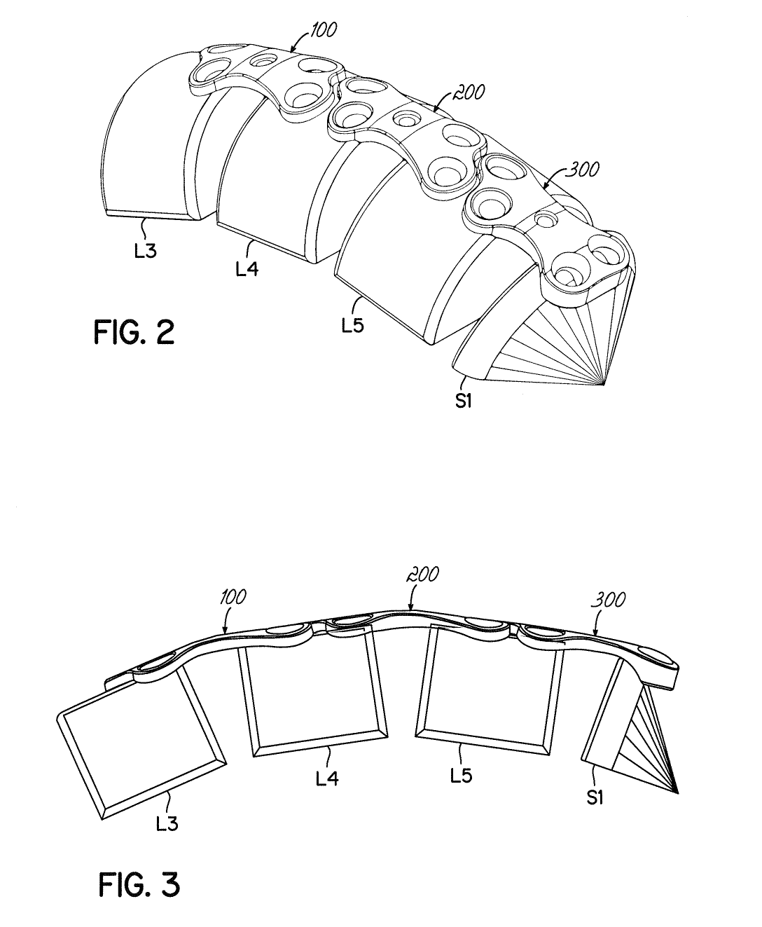

FIGS. 2 and 3 are anterior-posterior schematic views of a portion of the spinal column to which a plurality of fixation plates 100, 200, and 300 are implanted according to one embodiment of the present invention. As shown in FIG. 2, the fixation plate 100 is implanted across the vertebrae L3 and L4, the fixation plate 200 is implanted across the vertebrae L4 and L5, and the fixation plate 300 is implanted across the vertebrae L5 and S1. As shown in FIG. 3, the fixation plates 100, 200, and 300 may be configured to generally conform to the both the overall curvature of the patient's spine as well as the localized shape of the vertebrae to which they are attached. In the illustrated embodiment, the fixation plates 100, 200, and 300 each have a substantially uniform thickness along their respective lengths. In other embodiments, as shown and discussed in detail below, the fixation plates according to the present invention may have non-uniform thicknesses. As will be apparent to those of ordinary skill in the art, the fixation plates 100, 200, and 300 of the present invention are readily adaptable for implantation in other regions of the spinal column.

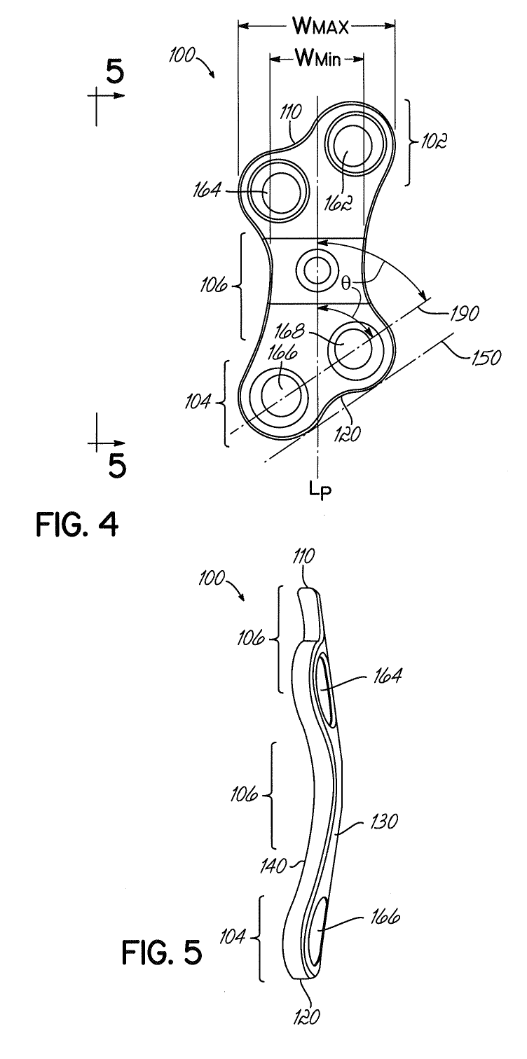

FIGS. 4 and 5 depict anterior and side views, respectively, of the spinal fixation plate 100 according to one embodiment of the present invention. As shown in FIGS. 4 and 5, the fixation plate 100 includes an upper end portion 102, a lower end portion 104, a middle portion 106, a top surface 130 and a bottom surface 140. The upper portion includes an upper edge 110 and the lower portion includes a lower edge 120. As shown, the bottom surface 140 are spaced apart from the top surface 130 by the upper and lower edges 110, 120. The fixation plate 100 defines a longitudinal axis Lp. At least one of the upper and lower edges 110, 120 in this embodiment may be generally oblique to the longitudinal axis Lp and generally parallel to a line 150, which is at an angle θ from the longitudinal axis Lp. In this illustrative embodiment, e is about 60°. In other embodiments, the angle θ may be from about 40° to about 80°. Placement of one or more of the upper and lower edges 100, 120 in a generally oblique position relative to the longitudinal axis Lp allows the fixation plate 100 to be placed closer to another fixation plate 200, as is further discussed below. As is also further shown below, the fixation plate 100 is configured for fixation to the anterior spine.

The fixation plate 100 further has apertures 162, 164, 166, 168 for receiving bone screws (not shown) for affixing the fixation plate 100 to the target vertebrae to be fused, as shown and discussed in more detail below. The apertures 162, 164 or 166, 168 near each edge 110 or 120 are positioned along a line parallel to a line 190 that is generally parallel to the line 150 and, accordingly, is also generally oblique to the longitudinal axis Lp of the fixation plate 100. In such embodiments, the line 190 is also oriented at the angle θ from the longitudinal axis Lp, but can be oriented at other suitable angles as well.

As further shown, the lumber plate 100 has a width, which is at its maximum Wmax near the upper edge 110 and lower edge 120 and at its minimum Wmin at about the midpoint of the middle section 106. The wider end portions 102 and 104 allow placement of two bone screws at each end of the fixation plate 100, thereby ensuring a stable affixation of the fixation plate 100 to the vertebrae to be fused.

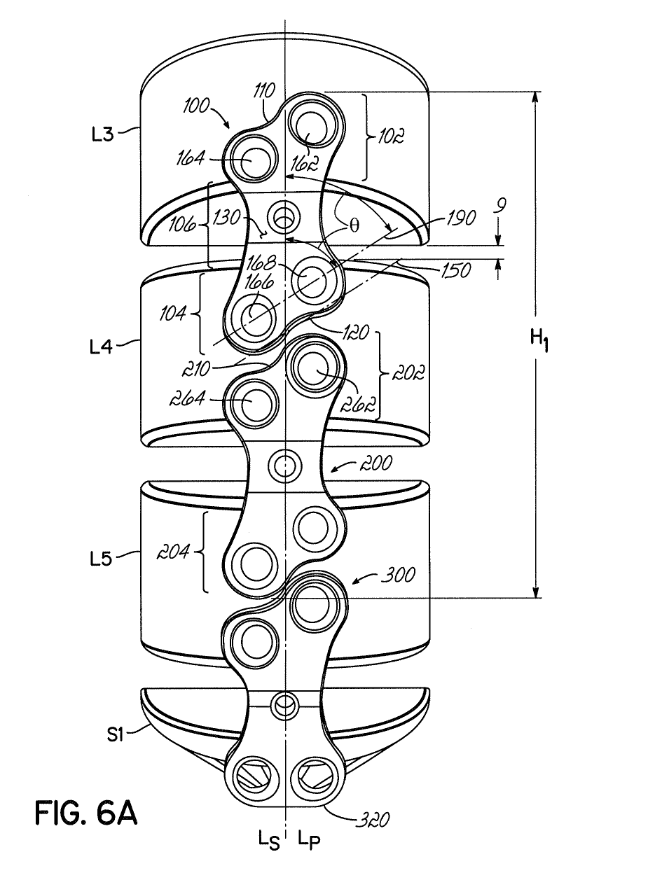

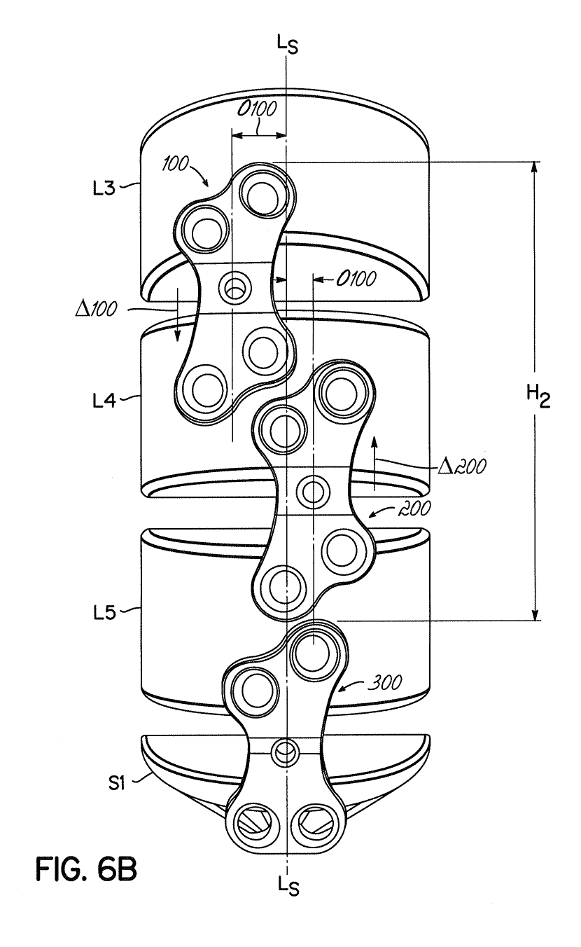

FIGS. 6(a) and 6(b) depict the fixation plate 100 implanted on the anterior spine to fuse a patient's L3 and L4 vertebrae (the vertebrae are shown schematically). As shown in FIG. 6(a), the fixation plate 100 may be positioned such that the upper apertures 162, 164 near the upper edge 110 allow bone screws to pass there through to affix the upper end portion 102 of the fixation plate 100 to the vertebra L3. Similarly, the fixation plate 100 is positioned such that the lower apertures 166, 168 near the lower edge 120 allow additional bone screws to pass them to affix the lower end portion 104 of the fixation plate 100 to the vertebra L4. As shown, the narrower middle portion 106 facilitates access to the disc space 9 between the adjacent vertebrae L3 and L4 during surgery. In the illustrated embodiment, the longitudinal axis Lp of the fixation plate 100 is generally aligned with the axis Ls of the patient's spine.

As further shown in FIG. 6(a), the fixation plates 200, 300 may be implanted in an end-to-end fashion together with the fixation plate 100. The fixation plate 200, which links the vertebrae L4 and L5, is identical to fixation plate 100 in the illustrated embodiment. In particular, upper end portion 202 and lower end portion 204 of the plate 200 are substantially identical to the upper end portion 102 and lower end portion 104, respectively, of the fixation plate 100. Additionally, the upper edge 210 of the lumber plate 200 is also generally oblique to the longitudinal axis Lp of the fixation plate 200 and generally parallel to the lower edge 120 of the fixation plate 100 when both fixation plates 100, 200 are implanted. Additionally, in this illustrative embodiment, the fixation plate 300 is used to stabilize the vertebrae L5 and S1 relative to each other. The fixation plate 300 in this example is otherwise similar to the other two fixation plates 100, 200 but has a lower edge 320 that is generally perpendicular to the longitudinal axis Lp.

Referring to FIG. 6(b), the fixation plates 100, 200 and 300 are shown implanted in a portion of the spine with the position of the fixation plates 100 and 200 changed as compared to their positions in FIG. 6(a). As shown in FIG. 6(b), the longitudinal axes of the fixation plates 100 and 200 have been offset from the spinal longitudinal axis Ls by an amount designated by O₁₀₀ and O₂₀₀, respectively. As further shown, the fixation plate 100 has been displaced inferiorily parallel to the longitudinal axis Ls as indicated by the arrow Δ₁₀₀, and the fixation plate has been displaced superiorily parallel to the longitudinal axis Ls as indicated by the arrow Δ₂₀₀, from their positions shown in FIG. 6(a).

Thus, as a result of this longitudinal displacement of the fixation plates 100 and 200, the total height H2 of the combination of fixation plates 100 and 200 can be smaller than its smallest value H1 when the longitudinal axes of the fixation plates 100, 200 are aligned as in FIG. 6(a). As will be apparent to those skilled in the art, the novel configuration of the end portions 102, 104, and 202 of the fixation plates 100, 200, permits this longitudinal displacement of the fixation plates 100, 200 while requiring a minimal amount of medial and/or lateral offset of the fixation plates 100, 200 from the longitudinal axis Ls. In general, the oblique edges 120, 210 allows an upward movement of the fixation plate 200 (and/or downward movement of the fixation plate 100) with an offset between the longitudinal axes of the fixation plates 100, 200 of less than half of the maximum width Wmax of the fixation plates 100, 200. In contrast, with traditional fixation plates lacking the novel end portion configuration of the illustrated embodiment of the present invention, the plates would have to be offset medially and/or laterally from the longitudinal axis Lp to a greater degree in order to adjust the position of the plates longitudinally, which may result in sub-optimal placement of the plates.

As further illustrated, offsetting the longitudinal positional adjustment of the fixation plates 100, 200 permits the bone screw apertures 262, 264 or 166, 168 to be positioned towards the midsection of the vertebra L4. Such movement is desirable as provides the surgeon with flexibility when placing bone screws.

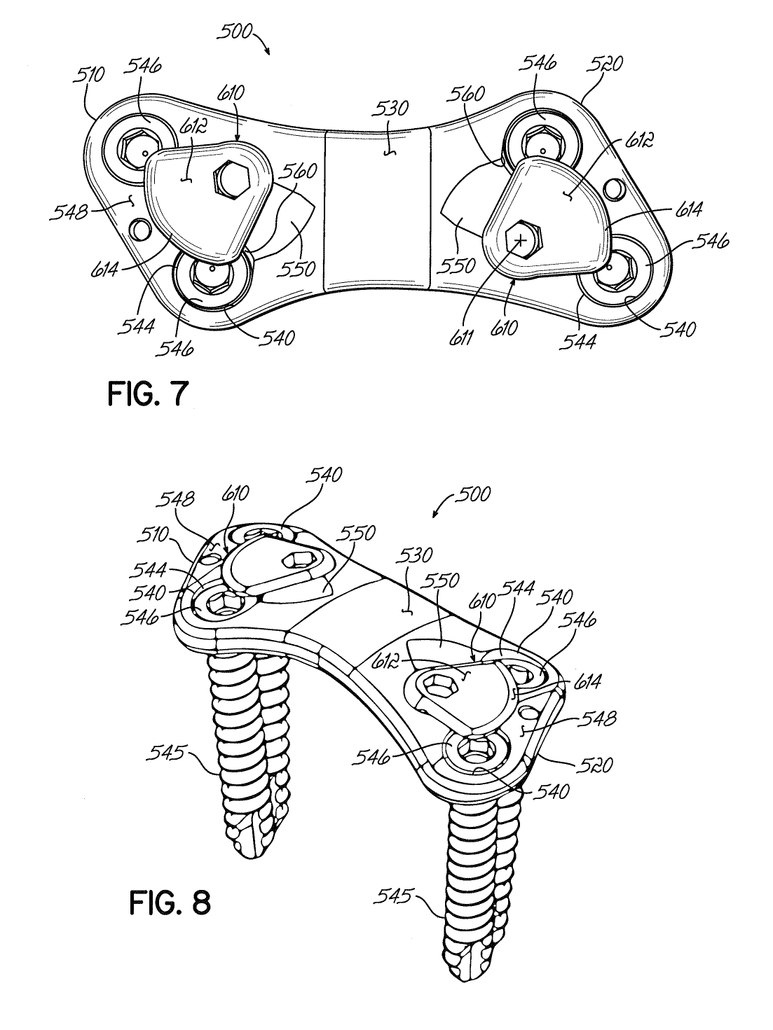

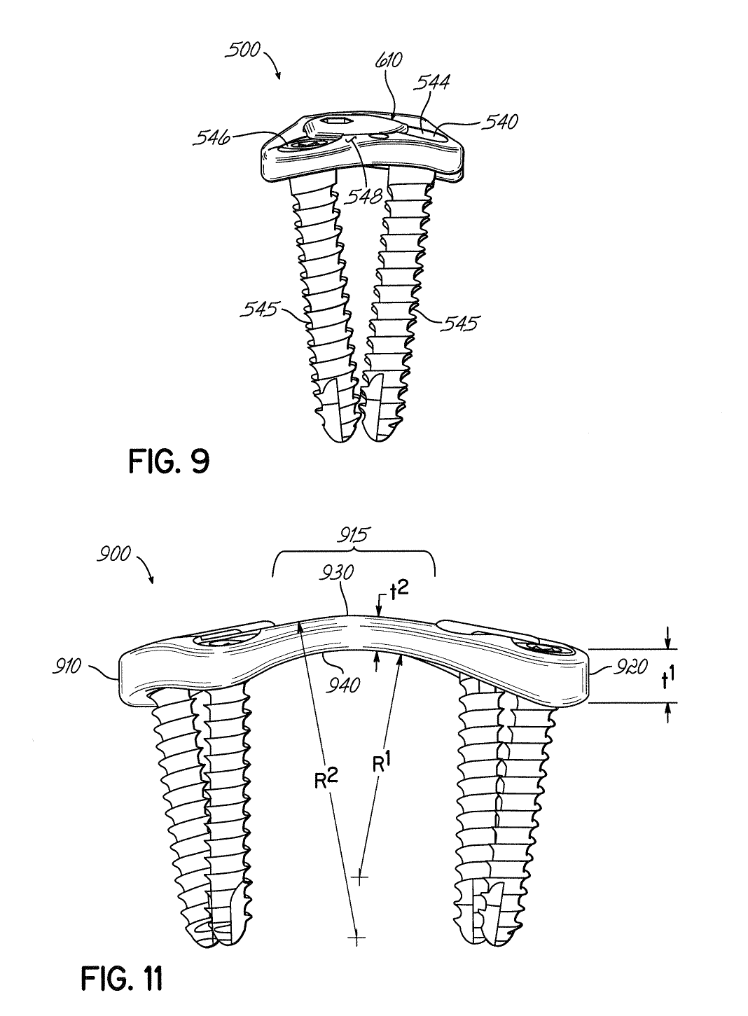

FIGS. 7-10(b) illustrate a fixation plate 500 according to a second embodiment of the invention. As shown in FIGS. 7-10(b), the fixation plate 500 may include an upper portion 510, a lower portion 520, and a top surface 530. Each of the upper and lower portions 510 and 520 may include at least one aperture 540 having a countersunk portion 544 near the top surface 530. As shown, a bone screw 545 having a screw head 546 may be inserted partially through each aperture 540, with the screw head 546 positioned in the countersunk portion 544 such that the screw head 546 does not extend above the top portion 530. As will be readily understood by those skilled in the art, the bone screws 545 can be screwed and partially implanted into the vertebrae (e.g., L3 and L4 in FIGS. 1 and 3) to facilitate attachment of the fixation plate 500 to the vertebrae.

As illustrated, the top portion 530 may include, in both the upper and lower ends 510 and 520, a curved region 548 and a generally flat region 550, with a step 560 between the curved and generally flat regions 548 and 550. Additionally, each portion 510 and 520 may include a locking member 610 pivotally attached to the top surface 530 at a pivot point 611. As shown, the locking member 610 may be disposed within the flat region 550.

In the illustrated embodiment, the locking member 610 can be positioned in at least a locking location, as shown in FIGS. 7-10(b), in which the locking member 610 is positioned above the screw heads 546 of the bone screws 545 when the screw heads 546 are positioned in the countersunk portions 544 of the apertures 540. In its locking location, the locking member 610 prevents, or at least impedes, the outward motion of the bone screws 545 from their implanted positions in the vertebrae.

The locking member 610 can further be pivotally displaced into at least a second, unlocking location (not illustrated), in which the locking member 610 does not cover the recesses 544 or the apertures 540, and accordingly, does not cover the screw heads 546. Thus, in the unlocking location, the locking member 610 permits bone screws 545 to be implanted into, or withdrawn from, the vertebra through the fixation plate 500.

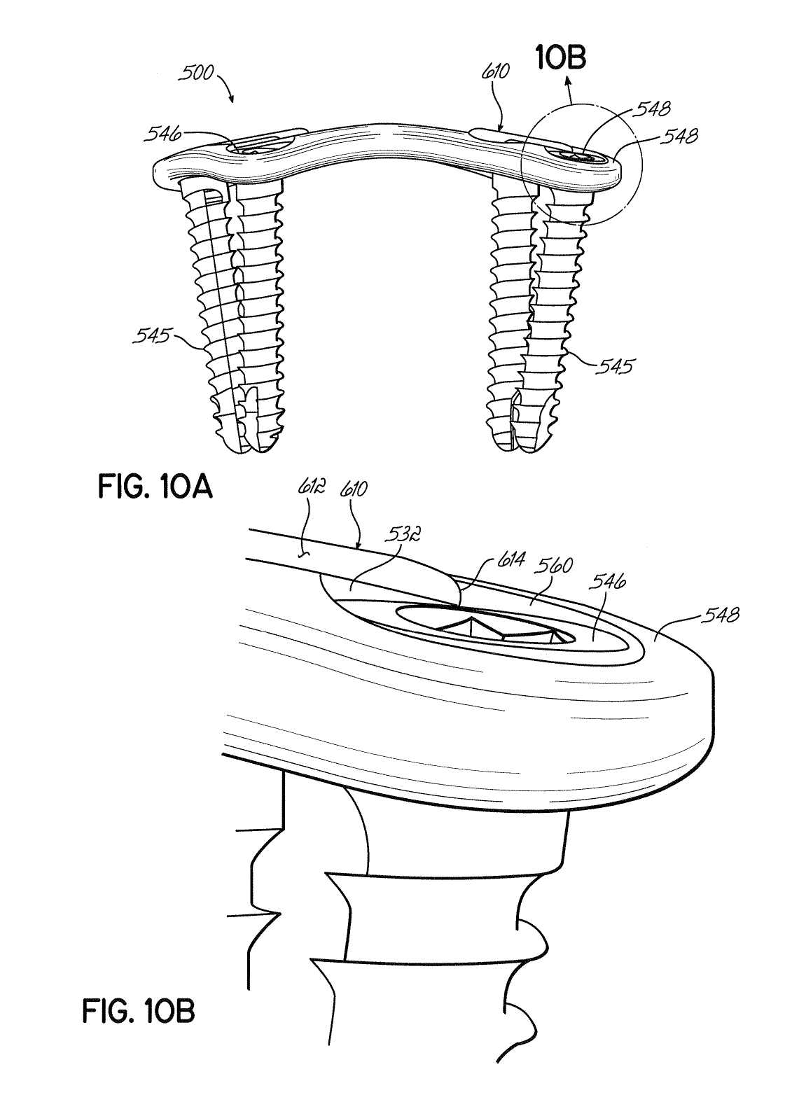

As illustrated, the locking member 610 may further include a locking member upper surface 612 having a beveled portion 614. As most clearly illustrated in FIGS. 10(a) and 10(b), and in particular, in the enlarged Detail ‘A’ in FIG. 10(b), the step 560 between the flat region 550 and the curved region 548 may be configured such that when the locking member 610 is swung into the locking location, at least the beveled tip portion 614 is generally flush with the top surface 530.

FIG. 11 shows a fixation plate 900 according to another embodiment of the present invention. As shown in FIG. 11, the fixation plate 900 may include an upper edge 910, a lower edge 920, and a middle portion 915 there between. In addition, the fixation plate 900 may have a top surface 930 and a bottom surface 940. The fixation plate 900 further has a thickness t1 near the upper and lower edges 910, 920, and a thickness t2 near or in the middle portion 915. In the illustrated embodiment, the thickness t1 is greater than the thickness t2. The greater thickness t1 near the upper and lower edges 910 and 920 permits the fixation plate 900 to effectively conform to non-uniform vertebral shapes, such as those that may result from a malformation or disease, and thereby can perform a bone defect-filling function. For example, for a patient from whom a portion of a vertebra has been removed as a result of, for instance, cancer surgery, the thickness t1 of the fixation plate 900 can be selected to substantially fill the cavity created by the removal of vertebral material.

As further illustrated in FIG. 11, as a result of this difference in the thicknesses t1 and t2, the bottom surface 940 has a curvature with a smaller radius of curvature R1 than the radius of curvature R2 of the top surface 930. As will be recognized by those skilled in the art, the individual vertebrae (e.g., L3 and L4 in FIG. 1) may have a relatively pronounced localized curvature in the areas of attachment of the fixation plate 900 as compared to the overall curvature of the spinal column. Accordingly, in the embodiment illustrated in FIG. 11, the fixation plate 900 may be configured such that the radius of curvature R1 of the bottom surface 940 substantially matches the relatively pronounced localized curvatures of the vertebral portions covered by the fixation plate 900 when it is affixed to the spine. Additionally, the radius of curvature R2 of the top surface 930 in this embodiment may more closely match the overall curvature of the portion of the spine onto which the fixation plate 900 is affixed.

FIG. 12 shows a fixation plate 1000 according to another embodiment of the present invention. As shown in FIG. 12, the fixation plate 1000 may have an upper edge 1010, a middle portion 1015, and a lower edge 1020. A further shown, the fixation plate 1000 may have a thickness t3 near the upper and lower edges 1010 and 1020 and a thickness t4 near or in the middle portion 1015. The fixation plate 1000 may be substantially similar to the fixation plate 900 discussed above except that the difference between the thickness t3 and the thickness t4 is more pronounced than the difference between the thicknesses t1 and t2 in the embodiment of the fixation plate 900 illustrated in FIG. 11. Accordingly, the fixation plate 1000 can accommodate a vertebral structure in which a larger portion of a vertebra to be stabilized has been removed or is otherwise missing. Thus, the fixation plates 900 and/or 1000 according to the present invention can be provided thicknesses with a wide range of thicknesses and configurations to suit the needs of a particular patient.

Although the illustrative embodiments described above involve fixation plates, similar devices applicable to other portions of the spine are also within the scope of the invention. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified, and the scope of the present invention is intended to embrace all such alternatives, modifications, permutations and variations as fall within the scope of the claims, together with all equivalents thereof.

1. An orthopedic fixation device for attachment to at least two separate vertebrae of a patient's spinal column, the device comprising:
a plate defining a longitudinal axis, the plate configured to be affixed to the at least two separate vertebrae with the longitudinal axis generally aligned with the longitudinal axis of the spinal column, wherein the plate comprises:
an upper edge and a lower edge positioned apart along the longitudinal axis wherein at least one of the edges is oriented generally oblique to the longitudinal axis; and
a top surface and a bottom surface separated by the upper and lower edges.

2. The orthopedic fixation device of claim 1 wherein the plate further defines a first aperture proximate the upper edge and a second aperture proximate the lower edge, wherein the first and second apertures have a size and shape suitable for receiving a first bone screw and a second bone screw at least partially therethrough, respectively, and wherein the apertures are spaced a sufficient distance apart to permit the bone screws to be implanted in the two separate vertebrae through the respective apertures.

3. The orthopedic fixation device of claim 2 wherein the plate further defines a third aperture proximate one of the upper or lower edges, wherein the third aperture has a size and shape and is positioned to receive a third bone screw at least partially therethrough to be implanted into one of the two separate vertebrae.

4. The orthopedic fixation device of claim 3 wherein the plate defines a fourth aperture proximate to a different one of the upper or lower edges than the third aperture and having a size and shape to receive a fourth bone screw at least partially therethrough.

5. The orthopedic fixation device of claim 3 wherein the two apertures proximal to the same one of the upper or lower edges are positioned along a line at a non-perpendicular angle from the longitudinal axis.

6. The orthopedic fixation device of claim 5 wherein the angle is from about 40 degrees to about 80 degrees.

7. The orthopedic fixation device of claim 4 wherein the two apertures proximal to the upper edge are positioned along a first line oriented at a non-perpendicular angle from the longitudinal axis, and wherein the two apertures proximate the lower edge are positioned along a second line oriented at a non-perpendicular angle from the longitudinal axis.

8. The fixation device of claim 1 wherein the plate has a non-uniform thickness along the longitudinal axis.

9. The orthopedic fixation device of claim 8 wherein the bottom surface comprises a portion configured to be more closely matching in shape the vertebral portion covered by the portion of the bottom surface than the corresponding top surface when the fixation device is affixed to the spine.

10. The orthopedic fixation device of claim 8 wherein the top surface is curved along the longitudinal axis with a curvature substantially the same as a curvature of a portion of the spinal column to which the orthopedic fixation device is affixed.

11. The orthopedic fixation device of claim 8 wherein the plate has a greater thickness at the upper and lower edges than at a portion between the upper and lower edges along the longitudinal axis.

12. The orthopedic fixation device of claim 1 wherein the plate has a width in a direction substantially perpendicular to the longitudinal axis, and wherein the width is non-uniform along the longitudinal axis such that the width is smaller in a middle portion of the plate than in portions more proximate the upper and lower edges, respectively.

13. The orthopedic fixation device of claim 2 further comprising a locking member pivotally coupled to the plate and adapted to at least partially cover at least one of the first or second apertures by an amount sufficient to impede withdrawal of the first or second bone screws after implantation in a vertebra through the first or second aperture, respectively.

14. The orthopedic fixation device of claim 13 wherein the locking member is positionable in a locking position and an unlocking position, and wherein the locking member is adapted to permit the at least one of the first or second bone screws to be implanted into or withdrawn from the vertebra when the locking member is in the unlocking position and to impede the at least one of the first or second bone screws from withdrawing from the vertebra when the locking member is in the locking position.

15. The orthopedic fixation device of claim 14 wherein the locking member is pivotally attached to the plate and pivotally positionable between the locking and unlocking positions.

16. The orthopedic fixation device of claim 15 wherein the locking member has a top surface portion that is substantially flush with the top surface of the plate.

17. The orthopedic fixation device of claim 3 further comprising a locking member pivotally coupled to the plate and pivotally positionable between a locking position and an unlocking position, wherein the locking member is adapted to permit the bone screws passing through the two apertures proximate the same one of the upper or lower edges to be implanted into or withdrawn from a vertebra when the locking member is in the unlocking position, and to impede the bone screws from withdrawing from the vertebra when the locking member is in the locking position.

18. A method of vertebral fixation comprising:
affixing to at least one pair of vertebrae in a spinal column an orthopedic fixation device, wherein the orthopedic fixation device includes a plate defining a longitudinal axis and a transverse axis and further comprises:
an upper edge and a lower edge positioned apart along the longitudinal axis wherein at least one of the edges is oriented generally oblique to the longitudinal axis, and
a top surface and a bottom surface separated by the upper and lower edges;
wherein the longitudinal axis of the plate is oriented generally parallel to the spinal column.

19. The method of claim 18 wherein the affixing step further comprises:
affixing a first and second vertebrae relative to each other using a first of the plurality of fixation devices; and
affixing the second and a third vertebrae relative to each other using a second of the plurality of fixation devices,
with the longitudinal axes of the plates of the first and second fixation devices are offset from each other.

20. The method of claim 19 wherein the plates of the first and second fixation devices have a maximum width, and the offset between the longitudinal axes is less than one-half of the maximum width of the plates, and wherein the affixing step further comprises positioning the plates such that a total height of the plates along the spinal column is less than the minimum total height attainable with the longitudinal axes of the plates aligned with each other.

21. The method of claim 18 wherein a first portion of the plate has a greater thickness than a second portion of the plate, the method further comprising:
matching the first and second portions of the plate with appropriate vertebral portions of the spinal column.

22. An orthopedic fixation device for attachment to at least two separate vertebrae of a patient's spinal column, the device comprising:
a plate defining a longitudinal axis, the plate configured to be affixed to the at least two separate vertebrae with the longitudinal axis generally aligned with the longitudinal axis of the spinal column;
wherein the plate has a non-uniform thickness along the longitudinal axis;
an upper edge of the plate and a lower edge of the plate positioned apart along the longitudinal axis; and
a top surface of the plate and a bottom surface of the plate separated by the upper and lower edges.

23. The orthopedic fixation device of claim 22 wherein the plate further defines a first aperture proximate the upper edge and a second aperture proximate the lower edge, wherein the first and second apertures have a size and shape suitable for receiving a first bone screw and a second bone screw at least partially therethrough, respectively, and wherein the apertures are spaced a sufficient distance apart to permit the bone screws to be implanted in the two separate vertebrae through the respective apertures.

24. The orthopedic fixation device of claim 22 wherein the bottom surface comprises a portion configured to be more closely matching in shape the vertebral portion covered by the portion of the bottom surface than the corresponding top surface when the fixation device is affixed to the spine.

25. The orthopedic fixation device of claim 22 wherein the top surface is curved along the longitudinal axis with a curvature substantially the same as a curvature of a portion of the spinal column to which the orthopedic fixation device is affixed.

26. The orthopedic fixation device of claim 22 wherein the plate has a greater thickness at the upper and lower edges than at a portion between the upper and lower edges along the longitudinal axis.
